(19) United States
(12) Patent Application Publication
(10) Pub. No.: US 2022/0068469 A1
(43) Pub. Date: Mar. 3, 2022

(54) MEDICAL IMAGE PROCESSING APPARATUS, SYSTEM, AND METHOD

(71) Applicant: CANON MEDICAL SYSTEMS CORPORATION, Otawara-shi (JP)

(72) Inventors: Takuya SAKAGUCHI, Utsunomiya (JP); Kazumasa ARAKITA, Utsunomiya (JP); Hideaki ISHII, Nasushiobara (JP); Takahiko NISHIOKA, Otawara (JP)

(73) Assignee: CANON MEDICAL SYSTEMS CORPORATION, Otawara-shi (JP)

(21) Appl. No.: 17/459,204

(22) Filed: Aug. 27, 2021

(30) Foreign Application Priority Data
Aug. 27, 2020 (JP) 2020-143280

(51) Int. Cl. G16H 30/40; G16H 50/30; G06T 7/00

(52) U.S. Cl. CPC G16H 30/40; G16H 50/30; G06T 2207/30048; G06T 2207/10081; G06T 7/0012

(57) ABSTRACT

A medical image processing apparatus according to an embodiment includes processing circuitry. The processing circuitry acquires fractional flow reserve at rest in a coronary artery of a subject, and fractional flow reserve at stress in the coronary artery. The processing circuitry calculates an index value based on comparison between the fractional flow reserve at rest and the fractional flow reserve at stress. The processing circuitry displays the index value as an index for a myocardial function of the subject.

### CROSS-REFERENCE TO RELATED APPLICATIONS

This application is based upon and claims the benefit of priority from Japanese Patent Application No. 2020-143280, filed on Aug. 27, 2020; the entire contents of which are incorporated herein by reference.

### FIELD

Embodiments described herein relate generally to a medical image processing apparatus, system, and method.

### BACKGROUND

Myocardial ischemia including angina of effort is a disease occurring in an enormous number of patients as a chronic disease associated with an aging population. As a testing method/index of the myocardial ischemia, there is known coronary flow reserve (CFR), which is measured by giving exercise stress or drug stress.

For the exercise stress, for example, measurement using a treadmill and echo is usually performed. For the drug stress, for example, there is known a method of measuring the CFR by calculating a myocardial blood flow (MBF) by measuring CT myocardial perfusion (CTP) in a stress state obtained by administration of adenosine to a subject by using computed tomography (CT), and comparing the MBF with a value in a state obtained by no administration of adenosine.

For the drug stress, there is also known a method of measuring the CFR by, for instance, inserting a flow wire as a flowmeter into a coronary artery during treatment, measuring a blood flow velocity at adenosine stress and a blood flow velocity at no stress, and comparing the blood flow velocities.

For example, in the measurement of the CFR using the CT, the myocardial blood flow (MBF) at stress is calculated by image analysis of image data obtained by performing photographing using a CTP protocol in a stress state obtained by administration of adenosine (adenosine stress state, also referred to as hyperemia state) in order to increase oxygen uptake of myocardial cells. Similarly, the myocardial blood flow (MBF) at rest is calculated by similarly collecting and analyzing image data in a rest state (normal state obtained by administrating no drug). In the measurement of the CFR using the CT, the CFR is acquired by calculating a ratio between the myocardial blood flow (MBF) at stress and the myocardial blood flow (MBF) at rest.

### BRIEF DESCRIPTION OF THE DRAWINGS

FIG. 1 is a view illustrating a configuration example of a medical image processing system and a medical image processing apparatus according to a first embodiment;

FIG. 2 is a view for explaining a relation between a myocardial function and an INDEX according to the first embodiment;

FIG. 3A is a view illustrating an example of display by a display control function according to the first embodiment;

FIG. 3B is a view illustrating an example of display by the display control function according to the first embodiment;

FIG. 4 is a view illustrating an example of display by the display control function according to the first embodiment;

FIG. 5 is a view illustrating an example of display by the display control function according to the first embodiment; and

FIG. 6 is a flowchart illustrating processing steps of a process performed by respective processing functions provided for processing circuitry of the medical image processing apparatus according to the first embodiment.

### DETAILED DESCRIPTION

A medical image processing apparatus according to an embodiment includes processing circuitry. The processing circuitry acquires fractional flow reserve at rest in a coronary artery of a subject, and fractional flow reserve at stress in the coronary artery. The processing circuitry calculates an index value based on comparison between the fractional flow reserve at rest and the fractional flow reserve at stress. The processing circuitry displays the index value as an index for a myocardial function of the subject.

Hereinafter, embodiments of a medical image processing apparatus, system, and method will be described in detail with reference to the drawings. It should be noted that the embodiments described below are not intended to limit a medical image processing apparatus, a medial image processing system and a medical image processing method according to the present application. Additionally, the embodiments can be combined with another embodiment or a conventional technique within a range not producing inconsistency in processing contents.

### First Embodiment

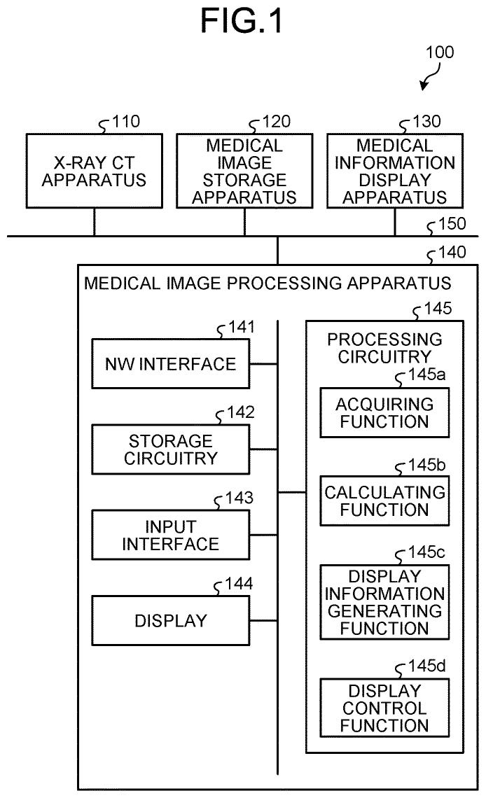

FIG. 1 is a view illustrating a configuration example of a medical image processing system and a medical image processing apparatus according to a first embodiment.

For example, as illustrated in FIG. 1, a medical image processing system 100 according to the present embodiment includes an X-ray computed tomography (CT) apparatus 110, a medical image storage apparatus 120, a medical information display apparatus 130, and a medical image processing apparatus 140. The respective apparatuses and systems are connected so as to be able to communicate with each other via a network 150.

In addition to the X-ray CT apparatus 110, the medical image processing system 100 may further include other medical image diagnostic apparatuses such as a magnetic resonance imaging (MRI) apparatus, an ultrasonic diagnostic apparatus, a positron emission tomography (PET) apparatus, and a single photon emission computed tomography (SPECT) apparatus. Moreover, the medical image processing system 100 may further include other systems such as an electronic medical chart system, a hospital information system (HIS), and a radiology information system (RIS).

The X-ray CT apparatus 110 generates a CT image regarding a subject. More specifically, the X-ray CT apparatus 110 collects projection data representing a distribution of X rays transmitted through the subject by turning an X-ray tube and an X-ray detector along a circular track around the subject. The X-ray CT apparatus 110 generates a CT image based on the collected projection data.

The medical image storage apparatus 120 stores various medical images regarding the subject. More specifically, the medical image storage apparatus 120 acquires the CT image from the X-ray CT apparatus 110 via a network 160, and stores the CT image by storing it in storage circuitry within the medical image storage apparatus. The medical image storage apparatus 120 is achieved by, for instance, computer equipment such as a server and a workstation. The medical image storage apparatus 120 is also achieved by, for example, a picture archiving and communication system (PACS), and stores the CT image in a format based on digital imaging and communications in medicine (DICOM).

The medical information display apparatus 130 displays various medical information regarding the subject. More specifically, the medical information display apparatus 130 acquires medical information such as the CT image and a processing result of image processing from the medical image storage apparatus 120 via the network 150, and displays the medical information on a display within the medical information display apparatus. The medical information display apparatus 130 is achieved by, for instance, computer equipment such as a workstation, a personal computer, and a tablet terminal.

The medical image processing apparatus 140 performs various image processing regarding the subject. More specifically, the medical image processing apparatus 140 acquires the CT image from the X-ray CT apparatus 110 or the medical image storage apparatus 120 via the network 150, and performs various image processing by using the CT image. The medical image processing apparatus 140 is achieved by, for instance, computer equipment such as a server and a workstation.

For example, the medical image processing apparatus 140 includes a network (NW) interface 141, storage circuitry 142, an input interface 143, a display 144, and a processing circuitry 145.

The NW interface 141 controls transmission of various data transmitted and received between the medical image processing apparatus 140 and another apparatus connected thereto via the network 150, and communication therebetween. More specifically, the NW interface 141 is connected to the processing circuitry 145 to output data received from another apparatus to the processing circuitry 145 or transmit data outputted from the processing circuitry 145 to another apparatus. The NW interface 141 is achieved by, for instance, a network card, a network adapter, or a network interface controller (NIC).

The storage circuitry 142 stores various data and various computer programs. More specifically, the storage circuitry 142 is connected to the processing circuitry 145 to store data inputted from the processing circuitry 145 or read and output stored data to the processing circuitry 145. The storage circuitry 142 is achieved by, for instance, a semiconductor memory element including a random-access memory (RAM) and a flash memory, a hard disk, or an optical disk.

The input interface 143 receives input operations of various instructions and various information from a user. More specifically, the input interface 143 is connected to the processing circuitry 145 to convert the input operations received from the user to electric signals and output the electric signals to the processing circuitry 145. The input interface 143 is achieved by, for instance, a trackball, a switch button, a mouse, a keyboard, a touchpad that allows a user to perform an input operation by touching an operation surface, a touchscreen obtained by integrating a display screen and a touchpad, a noncontact input interface using an optical sensor, or a voice input interface. In the present specification, the input interface 143 is not limited to those including a physical operation component such as a mouse and a keyboard. Examples of the input interface 143 include an electric signal processing circuit that receives an electric signal corresponding to an input operation from external input equipment provided separately from the apparatus, and outputs the electric signal to a control circuit.

The display 144 displays various information and various data. More specifically, the display 144 is connected to the processing circuitry 145 to display various information and various data outputted from the processing circuitry 145. The display 144 is achieved by, for instance, a liquid crystal display, a cathode ray tube (CRT) display, an organic EL display, a plasma display, or a touch panel.

The processing circuitry 145 controls the entire medical image processing apparatus 140. For example, the processing circuitry 145 performs various processing according to the input operations received from the user via the input interface 143. For instance, data transmitted from another apparatus is inputted into the processing circuitry 145 from the NW interface 141, and the processing circuitry 145 stores the inputted data in the storage circuitry 142. For example, the processing circuitry 145 also outputs data inputted from the storage circuitry 142 to the NW interface 141 to thereby transmit the data to another apparatus. For example, the processing circuitry 145 also displays the data inputted from the storage circuitry 142 on the display 144.

The configuration example of the medical image processing system 100 and the medical image processing apparatus 140 according to the present embodiment has been described above. For instance, the medical image processing system 100 and the medical image processing apparatus 140 according to the present embodiment are installed in a medical facility such as a hospital and a clinic, and assist diagnosis, formulation of a treatment plan, or the like regarding heart disease, performed by a user such as a doctor.

The medical image processing system 100 and the medical image processing apparatus 140 according to the present embodiment provide an index for a myocardial function based on fractional flow reserve (FFR) values of a subject. More specifically, the medical image processing apparatus 140 provides an index for a myocardial function based on comparison between FFR at rest and FFR at stress.

The FFR is an index indicating a blood flow ratio between “a case in which a coronary artery has a lesion (e.g., a stenosis)” and “a case in which the coronary artery has no lesion (e.g., no stenosis)”, and is used as an index for checking whether myocardial ischemia, if any, is caused by the lesion. More specifically, before treatment of the coronary artery, a pressure is obtained by inserting a pressure wire into the coronary artery in a stress state obtained by administration of adenosine and measuring an intravascular pressure (blood pressure). The obtained pressure is converted to a blood flow based on a theoretical formula to calculate the FFR value. There is also known a method of calculating the FFR value based on a pressure measured by the pressure wire in a rest state obtained by no administration of adenosine.

As another method of measuring the FFR, a method of calculating the FFR value by analyzing image data collected using CT is also known. More specifically, the FFR value is calculated by analysis using a CT image collected from a subject at rest.

The FFR can be acquired in the medical image processing system 100 and the medical image processing apparatus 140 according to the present embodiment by employing any method described above. That is, the FFR values used for the index for the myocardial function in the present embodiment may be acquired using any method.

Hereinafter, the medical image processing apparatus 140 according to the present embodiment will be described in detail. Note that a case in which the FFR values are calculated using the CT image, and the index for the myocardial function is calculated based on the calculated FFR values will be described below.

For example, as illustrated in FIG. 1, the processing circuitry 145 of the medical image processing apparatus 140 executes an acquiring function 145a, a calculating function 145b, a display information generating function 145c, and a display control function 145d in the present embodiment. The processing circuitry 145 is an example of processing circuitry.

The processing circuitry 145 is achieved by, for instance, a processor. In this case, the above respective processing functions are stored in the storage circuitry 142 in the forms of computer programs executable by a computer. The processing circuitry 145 reads and executes the respective computer programs stored in the storage circuitry 142, thereby achieving the functions corresponding to the respective computer programs. In other words, the processing circuitry 145 has the respective processing functions illustrated in FIG. 1 in a state in which the processing circuitry 145 reads the corresponding computer programs.

Note that the processing circuitry 145 may be composed of a plurality of independent processors combined together to achieve the respective processing functions with the respective processors executing the computer programs. Additionally, the respective processing functions of the processing circuitry 145 may be achieved by being appropriately dispersed or integrated in a single or a plurality of processing circuits. The respective processing functions of the processing circuitry 145 may be also achieved by combining hardware such as circuitry and software. Moreover, while the case in which the computer programs corresponding to the respective processing functions are stored in the single storage circuitry 142 has been described, the embodiments are not limited thereto. For example, the computer programs corresponding to the respective processing functions may be dispersedly stored in a plurality of memory circuits, and the processing circuitry 145 may read and execute the respective computer programs from the corresponding memory circuits.

The acquiring function 145a acquires the fractional flow reserve at rest in a coronary artery of the subject, and the fractional flow reserve at stress in the coronary artery. More specifically, the acquiring function 145a acquires a coronary artery CT image of the subject from the X-ray CT apparatus 110 or the medical image storage apparatus 120 via the NW interface 141. Here, the acquiring function 145a acquires a three-dimensional coronary artery CT image that can be used for calculating the FFR.

The acquiring function 145a then calculates the FFR at rest and the FFR at stress at each position of the coronary artery by a known method using computational fluid dynamics (CFD), artificial intelligence (AI), or the like, from the acquired coronary artery CT image of the subject.

For example, the acquiring function 145a acquires the FFR at rest and the FFR at stress by the computational fluid dynamics using the CT image including the coronary artery of the subject. In one example, the acquiring function 145a acquires the coronary artery CT image acquired from the subject at rest. The acquiring function 145a then calculates the FFR value at rest in a blood vessel target region by implementing the computational fluid dynamics by use of blood vessel shape data and analysis conditions based on the coronary artery CT image. The acquiring function 145a further estimates a stress state from the acquired coronary artery CT image, and calculates the FFR value at stress in the blood vessel target region by using the estimation result. Hereinafter, the FFR at rest is sometimes referred to as “Rest FFR”, and the FFR at stress as “Stress FFR”.

The calculating function 145b calculates an index value based on comparison between the FFR at rest and the FFR at stress. More specifically, the calculating function 145b calculates a ratio between the FFR at rest and the FFR at stress.

First, the definition of the FFR will be described. As described above, the FFR is defined by a ratio between a flow rate with no lesion and a flow rate with a lesion, and is calculated by the following formula (1). In the formula (1), “Qn” indicates a flow rate with no lesion (e.g., no stenosis), and “Qs” indicates a flow rate with a lesion (e.g., a stenosis).

$$F F R = \frac{Q s}{Q n} \qquad ( 1 )$$

The FFR is defined by an expression of dividing “Qs” by “Qn” as indicated in the formula (1). Here, a relation between a flow rate and a pressure in a blood vessel is brought into a proportional relation by obtaining a stress state by administration of adenosine to the subject, or targeting a predetermined period of a cardiac phase in a rest state. The FFR can be thereby replaced with the definition of a pressure. That is, the formula (1) can be expressed as the following formula (2) by bringing the relation between a flow rate and a pressure in a blood vessel into a proportional relation. In the formula (2), “Pa” indicates a pressure on the upstream side of the lesion (e.g., the stenosis), and “Pd” indicates a pressure on the downstream side of the lesion (e.g., the stenosis). Also, “Pv” indicates a pressure of the right atrium into which venous blood from the entire body flows.

$$F F R = \frac{Q s}{Q n} = \frac{P d - P v}{P a - P v} \qquad ( 2 )$$

For example, as indicated in the formula (2), “Qs” is expressed as “Pd-Pv”, and “Qn” as “Pa-Pv”. That is, the FFR is expressed by a ratio between values obtained by subtracting the baseline pressure of the blood vessel from the respective pressures on the upstream side and the downstream side of the lesion.

Since it can be considered that “Pa>>Pv” and “Pd>>Pv”, the formula (2) can be considered as indicated in the following formula (3).

$$F F R = \frac{Q s}{Q n} = \frac{P d - P v}{P a - P v} \approx \frac{P d}{P a} \qquad ( 3 )$$

That is, as indicated in the formula (3), the FFR is calculated by an expression of dividing “Pd” by “Pa”. For example, the acquiring function 145a calculates the “Rest FFR” and the “Stress FFR” at each position in the blood vessel target region from “Pa” and “Pd” by using the formula (3).

The calculating function 145b calculates the ratio between the “Rest FFR” and the “Stress FFR” calculated by the acquiring function 145a. For example, the calculating function 145b calculates an index “INDEX” for the myocardial function at each position in the blood vessel target region by dividing the “Stress FFR” by the “Rest FFR” as indicated in the following formula (4).

$$INDEX = \frac{Stress FFR}{Rest FFR} \qquad ( 4 )$$

As described above, the FFR is the index suggesting “whether myocardial ischemia, if any, is caused by a stenosis”. In current clinical practice, however, the FFR is used as “an index indicating a stenosis degree”, and a disorder regarding the myocardial function is not directly reflected therein. Thus, the calculating function 145b calculates the above “INDEX”, thereby calculating the index for the myocardial function that cannot be obtained only from the “Rest FFR” or from the “Stress FFR”.

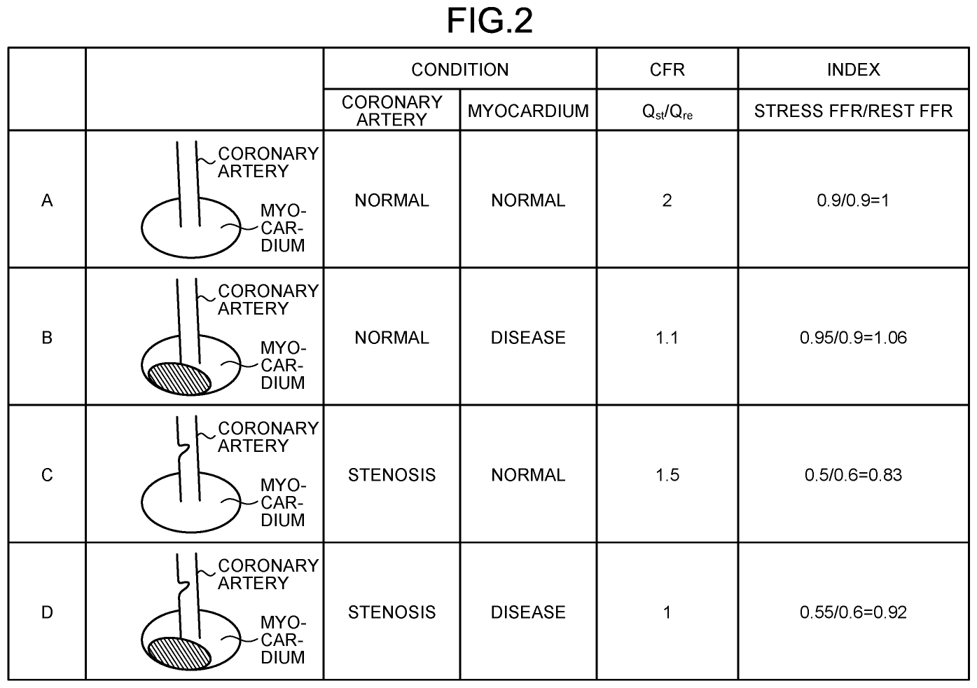

Hereinafter, a relation between the myocardial function and the INDEX will be described using FIG. 2. FIG. 2 is a view for explaining the relation between the myocardial function and the INDEX according to the first embodiment. FIG. 2 illustrates the CFR and INDEX of each of cases A to D associated with the conditions of the coronary artery and the myocardium. Note that each index value in FIG. 2 is a value described for the convenience of explanation, not an actually measured value.

For example, the “case A” indicates a case in which the coronary artery condition is “normal” and the myocardium condition is “normal” as illustrated in FIG. 2. In such a case, for instance, the “FFR” values are “0.9”, and the CFR (Qₛₜ/Qᵣₑ) value is “2”. When the coronary artery condition is “normal” and the myocardium condition is “normal”, the value of the “Stress FFR” and the value of the “Rest FFR” are not different. Thus, the “INDEX” value calculated by the “Stress FFR”/the “Rest FFR” is calculated as “0.9/0.9=1”.

Additionally, for example, the “case B” indicates a case in which the coronary artery condition is “normal” and the myocardium condition is “disease” as illustrated in FIG. 2. That is, the “case B” indicates a case in which the myocardial function is reduced. In such a case, for instance, the CFR (Qₛₜ/Qᵣₑ, ) value is “1”. This is because an increase in blood flow as in a healthy subject does not occur due to the reduction in myocardial function even when a stress state is obtained so as to increase the blood flow. Thus, the CFR (Qₛₜ/Qᵣₑ) value is decreased.

When the coronary artery condition is “normal” and the myocardium condition is “disease”, the “INDEX” value calculated by the “Stress FFR”/the “Rest FFR” is calculated as “0.95/0.9=1.06”. This is because the FFR values have such a property as to be more influenced by the blood flow in a stress state as compared to a rest state. Thus, the value of the “Stress FFR” changes by sensitively reacting to even an imperceptible decrease in myocardial blood flow. As a result, the “INDEX:1.06” in the “case B” indicates a value higher than the “INDEX:1” in the “case A”.

As described above, the “INDEX” according to the present embodiment is the index reflecting the condition of the myocardial function. The use of this index enables the medical image processing apparatus 140 according to the present embodiment to provide the index for the myocardial function without measuring the CFR value. For example, the condition of the “case B” (coronary artery: normal, myocardium: disease) cannot be diagnosed in conventional techniques unless the FFR value and the CFR value are individually measured. In the first place, the myocardium condition cannot be diagnosed when the value of only one of the “Stress FFR” (e.g., 0.95) or the “Rest FFR” (e.g., 0.9) is calculated.

Meanwhile, the medical image processing apparatus 140 according to the present embodiment can diagnose the condition of the “case B” (coronary artery: normal, myocardium: disease) only by calculating the “Stress FFR” and the “Rest FFR” and calculating the “INDEX”. That is, a user can diagnose the condition of the “case B” (coronary artery: normal, myocardium: disease) when the “FFR” values are within a normal range and the “INDEX” value exceeds a normal range.

As described above, the acquiring function 145a according to the present embodiment can acquire the “Stress FFR” and the “Rest FFR” from the coronary artery CT image acquired from the subject at rest. This enables the medical image processing apparatus 140 according to the present embodiment to easily calculate and provide the “INDEX” as the index for the myocardial function.

The medical image processing apparatus 140 according to the present embodiment can also diagnose whether the coronary artery has a disease at the same time.

For example, the “case C” indicates a case in which the coronary artery condition is “stenosis” and the myocardium condition is “normal” as illustrated in FIG. 2. In such a case, for instance, the CFR (Qst/Qre) value is “1.5”. Additionally, when the coronary artery condition is “stenosis” and the myocardium condition is “normal”, the value of the “Stress FFR” is “0.5”, and the value of the “Rest FFR” is “0.6”. Thus, the “INDEX” value calculated by the “Stress FFR”/the “Rest FFR” is calculated as “0.5/0.6=0.83”.

The user can thereby diagnose the condition of the “case C” (coronary artery: stenosis, myocardium: normal) by referring to the calculated result of the “FFR” (“0.5” and “0.6”) and the calculated result of the “INDEX” (“0.83”). That is, the user can diagnose the condition of the “case C” (coronary artery: stenosis, myocardium: normal) according to a state in which the “FFR” values are out of the normal range (for example, fall below a threshold set to “0.8”), and a degree of the “INDEX” value falling below the normal range.

Additionally, for example, the “case D” indicates a case in which the coronary artery condition is “stenosis” and the myocardium condition is “disease” as illustrated in FIG. 2. That is, the “case D” indicates a case in which the coronary artery has a stenosis and the myocardial function is reduced. In such a case, for instance, the value of the “Stress FFR” is “0.55”, the value of the “Rest FFR” is “0.6”, and the CFR (Qst/Qre) value is “1”. Note that the CFR value is maintained at about “1” since a mechanism “Auto Regulation” works to keep a constant blood flow even when the coronary artery has a stenosis and the myocardium has a disease.

When the coronary artery condition is “stenosis” and the myocardium condition is “disease”, the “INDEX” value calculated by the “Stress FFR”/the “Rest FFR” is calculated as “0.55/0.6=0.92”. The user can thereby diagnose the condition of the “case D” (coronary artery: stenosis, myocardium: normal) by referring to the calculated result of the “FFR” (“0.55” and “0.6”) and the calculated result of the “INDEX” (“0.92”). That is, the user can diagnose the condition of the “case D” (coronary artery: stenosis, myocardium: normal) according to a state in which the “FFR” values are out of the normal range (for example, fall below a threshold set to “0.8”), and a degree of the “INDEX” value deviating from the normal range (for example, a falling-below degree is smaller than that in the case C).

The display information generating function 145c generates various information to be displayed. More specifically, the display information generating function 145c generates an image to be displayed and reference information for diagnosis. For example, the display information generating function 145c three-dimensionally reconstructs the blood vessel region of the coronary artery in the coronary artery CT image to generate a three-dimensional image of the coronary artery. For instance, the display information generating function 145c generates a VR image, an SR image, a curved planar reconstruction (CPR) image, a multi planer reconstruction (MPR) image, a stretched multi planer reconstruction (SPR) image, or the like.

For example, the display information generating function 145c also generates information regarding treatment for the coronary artery and information regarding treatment for the myocardium as the reference information for diagnosis. More specifically, the display information generating function 145c discriminates the contents of treatment for the coronary artery and treatment for the myocardium based on the FFR values and the INDEX value, and generates information indicating the discrimination result.

The display control function 145d displays on the display 144 various information to be displayed, generated by the display information generating function 145c. More specifically, the display control function 145d displays the image to be displayed and the reference information for diagnosis on the display 144. The display control function 145d displays the INDEX calculated by the calculating function 145b as the index for the myocardial function of the subject.

For example, the display control function 145d compares the INDEX calculated at each position of the coronary artery of the subject with a threshold, identifies a position on the coronary artery where the INDEX falls below or exceeds the threshold, and displays a dominant region on the myocardium corresponding to the identified position. In one example, the display control function 145d identifies a position closest to a proximal where the INDEX falls below or exceeds the threshold in the positions of the coronary artery of the subject, and displays a dominant region on the myocardium corresponding to the identified position.

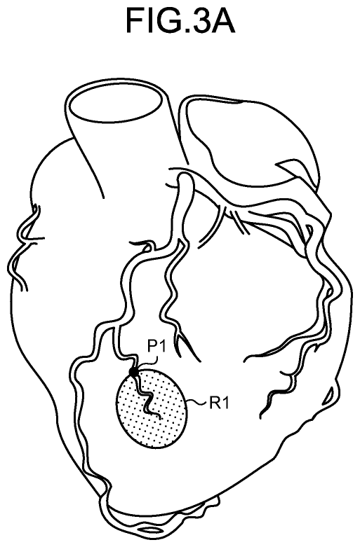

FIG. 3A is a view illustrating an example of display by the display control function 145d according to the first embodiment. For example, as illustrated in FIG. 3A, the display control function 145d compares the INDEX calculated at each position of the coronary artery of the subject with the threshold, identifies a position P1 on the coronary artery where the INDEX falls below or exceeds the threshold, and extracts a dominant region R1 to which blood is supplied by a distal blood vessel region relative to the identified position P1. Note that the dominant region is extracted by using a known method such as a Voronoi method.

The display control function 145d then displays on the display 144 a display image in which the dominant region R1 is displayed in a different color in the coronary artery CT image. The normal range of the INDEX is set to a value range, for instance, around “1.00”. In one example, the normal range of the INDEX is set to “0.95 to 1.05”.

The display control function 145d compares the INDEX calculated at each position of the coronary artery of the subject with the numerical value range, identifies a position on the coronary artery where the INDEX exceeds the numerical value range or a position on the coronary artery where the INDEX falls below the numerical value range, and displays display information based on the comparison result. More specifically, the display control function 145d displays disease candidates individually corresponding to the case in which the INDEX exceeds the numerical value range and the case in which the INDEX falls below the numerical value range. For example, the display control function 145d displays a disease candidate for the myocardium when the INDEX exceeds the numerical value range, and a disease candidate for the coronary artery when the index value falls below the numerical value range.

For instance, the display control function 145d determines that there is a disease possibility when the INDEX exceeds or falls below the set normal range “0.95 to 1.05”, and displays information based on the determination result. For example, the display control function 145d compares the INDEX value at each position of the coronary artery with the normal range “0.95 to 1.05”, and identifies a blood vessel position where the INDEX value falls below “0.95” and/or a blood vessel position where the INDEX value exceeds “1.05”.

Note that the normal range “0.95 to 1.05” is merely an example, and the normal range can be set to any value.

Additionally, the value for determining whether the INDEX is normal is not limited to the case of setting the range, and only two thresholds (e.g., 0.95 and 1.05) may be set.

Here, the display control function 145d can identify as the position P1 illustrated in FIG. 3A a position closest to a proximal where the INDEX falls below “0.95”, or a position closest to a proximal where the INDEX exceeds “1.05”. As described using FIG. 2, the INDEX indicates a high value when the myocardium has a disease, and indicates a low value when the coronary artery has a disease. That is, when the INDEX exceeds the normal range, a disease possibility in the myocardium is suggested. When the INDEX falls below the normal range, a disease possibility in the coronary artery (a decrease in blood supply function) is suggested.

Thus, the display control function 145d changes presentation information between the case in which the INDEX falls below the normal range and the case in which the INDEX exceeds the normal range. For example, when the INDEX falls below the normal range “0.95 to 1.05”, the display control function 145d identifies the position P1 closest to the proximal where the INDEX falls below “0.95”, extracts the dominant region R1 of the distal blood vessel region relative to the position P1, and displays the dominant region R1 in a different color as a dominant region having a disease possibility as illustrated in FIG. 3A. The display control function 145d can also display the candidate for the disease name occurring in the myocardium as well.

Meanwhile, when the INDEX exceeds the normal range “0.95 to 1.05”, the display control function 145d identifies the position P1 closest to the proximal where the INDEX exceeds “1.05”, and highlights a blood vessel branch including the position P1 as a blood vessel branch having a disease possibility. The display control function 145d can also extract the dominant region R1 for the position P1 similarly to the case in which the INDEX falls below the normal range, and display the dominant region R1 in a different color as a region possibly influenced by the disease of the coronary artery. The display control function 145d can also display the candidate for the disease name occurring in the coronary artery as well.

In the above embodiment, the case in which the single threshold is used for each of the upper limit and the lower limit has been described. However, the display control function 145d can use a plurality of thresholds for each of the upper limit and the lower limit. For example, “1.05” and “1.10” may be used as the threshold for determining the upper limit. In such a case, the display control function 145d compares the INDEX value at each position of the coronary artery with “1.05” and “1.10”, and identifies the blood vessel position where the INDEX value exceeds “1.05” and a blood vessel position where the INDEX value exceeds “1.10”.

Here, the display control function 145d identifies the position closest to the proximal where the INDEX exceeds “1.05”, and a position closest to a proximal where the INDEX exceeds “1.10” similarly to the above example. The display control function 145d then extracts the dominant region of the distal blood vessel region relative to the position closest to the proximal where the INDEX exceeds “1.05”, and a dominant region of a distal blood vessel region relative to the position closest to the proximal where the INDEX exceeds “1.10”, and displays the respective dominant regions in different colors as the dominant region having a disease possibility.

FIG. 3B is a view illustrating an example of display by the display control function 145d according to the first embodiment. For example, as illustrated in FIG. 3B, the display control function 145d identifies the position P1 closest to the proximal where the INDEX exceeds the threshold “1.05”, and a position P2 closest to a proximal where the INDEX exceeds “1.10”.

The display control function 145d then extracts the dominant region R1 of the distal blood vessel region relative to the position P1 closest to the proximal where the INDEX exceeds “1.05”, and a dominant region R2 of a distal blood vessel region relative to the position P2 closest to the proximal where the INDEX exceeds “1.10”, and displays the respective dominant regions in different colors as the dominant region having a disease possibility. For instance, the display control function 145d displays that the dominant region R2 has a higher disease possibility than the dominant region R1.

Similarly, for example, “0.95” and “0.9” may be used as the threshold for determining the lower limit. In such a case, the display control function 145d compares the INDEX value at each position of the coronary artery with “0.95” and “0.90”, and identifies the blood vessel position where the INDEX value falls below “0.95” and a blood vessel position where the INDEX value falls below “0.90”.

Here, the display control function 145d identifies the position closest to the proximal where the INDEX falls below “0.95”, and a position closest to a proximal where the INDEX falls below “0.90” similarly to the above example. The display control function 145d then highlights the region including the position closest to the proximal where the INDEX falls below “0.95”, and a region including the position closest to the proximal where the INDEX falls below “0.90” as the region having a disease possibility. For instance, the display control function 145d displays that the region including the position closest to the proximal where the INDEX falls below “0.90” has a higher disease possibility than the region including the position closest to the proximal where the INDEX falls below “0.95”.

The display control function 145d can also extract the dominant regions for the respective positions similarly to the case in which the INDEX falls below the normal range, and display the respective dominant regions in different colors as the region possibly influenced by the disease of the coronary artery. Here, the display control function 145d can also display the dominant region for the position closest to the proximal where the INDEX falls below “0.90” as a region more possibly influenced.

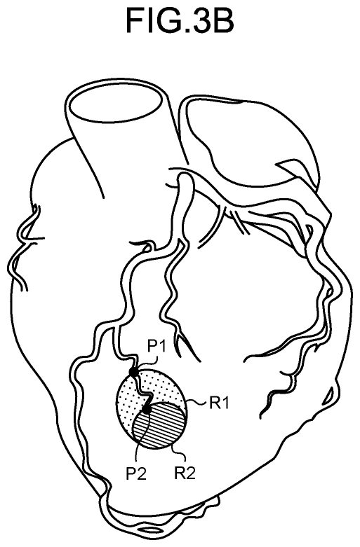

The display control function 145d can also display a warning when the area of the dominant region exceeds a threshold. FIG. 4 is a view illustrating an example of display by the display control function 145d according to the first embodiment. For example, the display control function 145d calculates the area of the dominant region R1 on the myocardium to which blood is supplied by the distal blood vessel region relative to the position P1 closest to the proximal where the INDEX exceeds the threshold, and compares the calculated area with the threshold. When the area of the dominant region R1 exceeds the threshold, the display control function 145d displays a warning “the size of the identified region exceeds a prescribed size” on the display 144 as illustrated in FIG. 4. When the position closest to the proximal where the INDEX exceeds the threshold is close to the distal of the blood vessel and the area of the dominant region does not exceed the threshold, healthy risk is low. Thus, the warning does not have to be displayed.

Similarly, the display control function 145d can calculate the area of the dominant region for the position closest to the proximal where the INDEX falls below the threshold, compare the calculated area with the threshold, and display the warning based on the comparison result.

While the above respective thresholds can be set to any values, the thresholds may be set according to the position of the coronary artery or the myocardium. For example, each threshold (normal range) for determining the abnormality of the INDEX may be set according to the position of the target coronary artery. For instance, a threshold for the coronary artery nourishing the myocardium corresponding to the left ventricle may be set to a value lower than the upper limit or a value higher than the lower limit. That is, in the coronary artery nourishing the myocardium corresponding to the left ventricle, the threshold (normal range) is set so as to detect an abnormality even when the INDEX value has a smaller change from a normal value.

Additionally, for example, each threshold compared with the area of the dominant region may be set according to the position of the dominant region. For instance, a threshold for the myocardium corresponding to the left ventricle may be set to a lower value. That is, when the identified dominant region is the myocardium corresponding to the left ventricle, the threshold is set so as to detect a high disease possibility even when the dominant region has a smaller area.

Moreover, the display control function 145d can display a combination of treatment for the coronary artery of the subject and treatment for the myocardium thereof based on a comparison result between the FFR at rest or the FFR at stress and a first threshold, and a comparison result between the INDEX and a second threshold. More specifically, the information indicating the discrimination result generated by the display information generating function 145c can be displayed on the display 144.

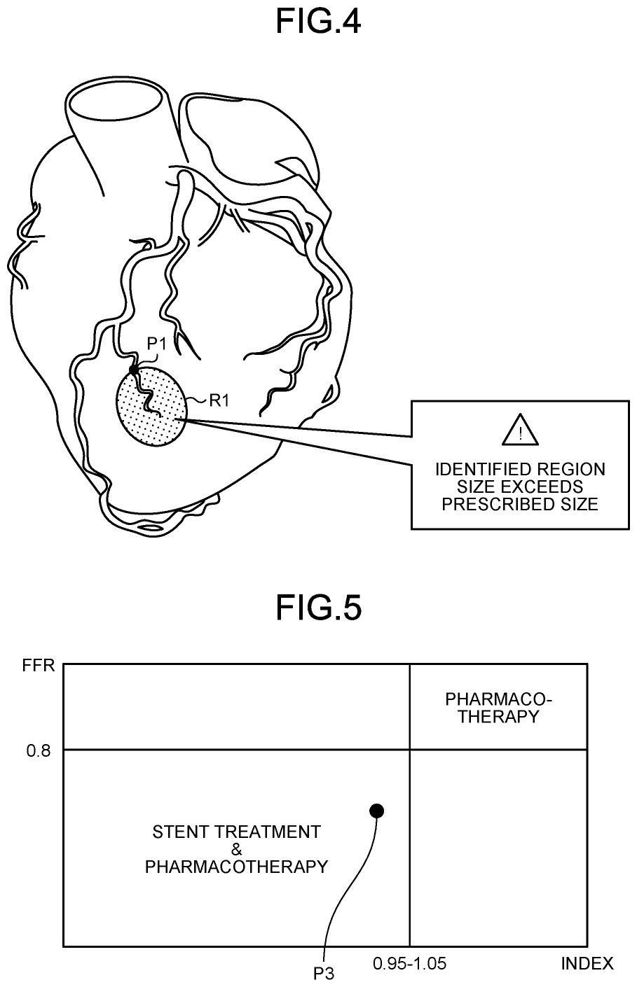

FIG. 5 is a view illustrating an example of display by the display control function 145d according to the first embodiment. For example, the display control function 145d displays information indicating a treatment plan based on the calculated results of the FFR and the INDEX on the display 144 as illustrated in FIG. 5. In one example, the display control function 145d displays information of a treatment plan proposing stent treatment of the coronary artery and pharmacotherapy of the myocardium when the FFR value is less than “0.8” and the INDEX value falls below the normal range as illustrated in FIG. 5.

Additionally, for example, the display control function 145d displays information of a treatment plan proposing pharmacotherapy of the myocardium when the FFR value is “0.8” or more and the INDEX value exceeds the normal range as illustrated in FIG. 5. Note that the information indicating the treatment plan illustrated in FIG. 5 is generated by the display information generating function 145c.

For instance, when the calculated FFR value is “0.6”, and the calculated INDEX value is “0.92”, the display control function 145d displays information of a treatment plan in which a marker is located at a position P3 corresponding to the calculated results as illustrated in FIG. 5. This enables the user to understand that the stent treatment and the pharmacotherapy are proposed as the treatment plan for the target subject.

Note that the information of the treatment plan illustrated in FIG. 5 is merely an example, and the embodiments are not limited thereto. For example, information of a treatment plan proposing stent treatment of the coronary artery may be displayed by further subdividing a region illustrated in FIG. 5.

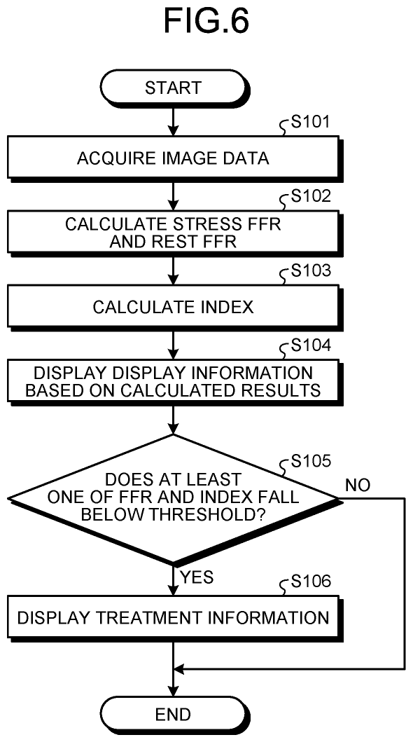

Next, processing steps of the medical image processing apparatus 140 will be described using FIG. 6. FIG. 6 is a flowchart illustrating the processing steps of a process performed by the respective processing functions provided for the processing circuitry 145 of the medical image processing apparatus 140 according to the first embodiment.

For example, as illustrated in FIG. 6, when receiving an instruction to start the process from a user via the input interface 143, the acquiring function 145a acquires a coronary artery CT image of a subject from the X-ray CT apparatus 110 or the medical image storage apparatus 120 (step S101). The acquiring function 145a further calculates “Stress FFR” and “Rest FFR” from the acquired coronary artery CT image of the subject (step S102). This process is achieved, for instance, by the processing circuitry 145 calling from the storage circuitry 142 and executing the computer program corresponding to the acquiring function 145a.

Subsequently, the calculating function 145b calculates an “INDEX” by using the “Stress FFR” and the “Rest FFR” calculated by the acquiring function 145a (step S103). This process is achieved, for instance, by the processing circuitry 145 calling from the storage circuitry 142 and executing the computer program corresponding to the calculating function 145b.

The display control function 145d then displays display information based on the calculated results (step S104), and determines whether at least one of the FFR and the INDEX falls below the threshold (step S105). When at least one of the FFR and the INDEX falls below the threshold (Yes at the step S105), the display control function 145d displays information regarding a treatment plan (step S106). When the FFR and the INDEX do not fall below the thresholds (No at the step S105), the display control function 145d does not display the information regarding a treatment plan. This process is achieved, for instance, by the processing circuitry 145 calling from the storage circuitry 142 and executing the computer program corresponding to the display control function 145d.

As described above, according to the first embodiment, the acquiring function 145a acquires the FFR at rest in the coronary artery of the subject, and the FFR at stress in the coronary artery. The calculating function 145b calculates the INDEX based on the comparison between the FFR at rest and the FFR at stress. The display control function 145d displays the INDEX as the index for the myocardial function of the subject. Thus, the medical image processing apparatus 140 according to the first embodiment can present the FFR indicating the low latitude of stenosis and the index for the myocardial function at the same time. For example, the “INDEX” is considered as an index reflecting a microcirculation influence of the myocardium. The medical image processing apparatus 140 can detect imperceptible micro vascular obstruction (MVO).

According to the first embodiment, the acquiring function 145a acquires the FFR at rest and the FFR at stress by the computational fluid dynamics using the medical image including the coronary artery of the subject. Thus, the medical image processing apparatus 140 according to the first embodiment can easily acquire the “Stress FFR” and the “Rest FFR”. The medical image processing apparatus 140 can also calculate the “Stress FFR” and the “Rest FFR” from the coronary artery CT image collected from the subject at rest. Consequently, the medical image processing apparatus 140 can calculate the index for the myocardial function without bringing the subject into a stress state. As compared to the case of calculating the CFR, the subject's burden can be greatly reduced.

According to the first embodiment, the calculating function 145b calculates the ratio between the FFR at rest and the FFR at stress. Thus, the medical image processing apparatus 140 according to the first embodiment can easily calculate the index for the myocardial function.

According to the first embodiment, the display control function 145d compares the INDEX calculated at each position of the coronary artery of the subject with the threshold, identifies the position on the coronary artery where the INDEX exceeds the threshold, and displays the dominant region on the myocardium corresponding to the identified position. Thus, the medical image processing apparatus 140 according to the first embodiment can present the region having the disease possibility in the myocardium.

According to the first embodiment, the display control function 145d identifies the position closest to the proximal where the INDEX exceeds the threshold in the positions of the coronary artery of the subject, and displays the dominant region on the myocardium corresponding to the identified position. Thus, the medical image processing apparatus 140 according to the first embodiment can present the entire myocardium region suspected to have the disease possibility.

According to the first embodiment, the display control function 145d displays the warning when the area of the dominant region exceeds the threshold. Thus, the medical image processing apparatus 140 according to the first embodiment can present that the myocardial function is greatly influenced.

According to the first embodiment, the display control function 145d compares the INDEX calculated at each position of the coronary artery of the subject with the numerical value range, identifies the position on the coronary artery where the INDEX exceeds the numerical value range or the position on the coronary artery where the INDEX falls below the numerical value range, and displays the display information based on the comparison result. Thus, the medical image processing apparatus 140 according to the first embodiment can display the information according to the manner of falling outside the numerical value range indicating the normal range.

According to the first embodiment, the display control function 145d displays the disease candidates individually corresponding to the case in which the INDEX exceeds the numerical value range and the case in which the INDEX falls below the numerical value range. Thus, the medical image processing apparatus 140 according to the first embodiment can display the information of the possible diseases according to the manner of falling outside the numerical value range indicating the normal range.

According to the first embodiment, the display control function 145d displays the disease candidate for the myocardium when the INDEX exceeds the numerical value range, and the disease candidate for the coronary artery when the INDEX falls below the numerical value range. Thus, the medical image processing apparatus 140 according to the first embodiment can determine the disease possibility in the myocardium and the disease possibility in the coronary artery based on the INDEX value, and display the information.

According to the first embodiment, the display control function 145d displays the combination of the treatment for the coronary artery of the subject and the treatment for the myocardium thereof based on the comparison result between the FFR at rest or the FFR at stress and the first threshold, and the comparison result between the INDEX and the second threshold. Thus, the medical image processing apparatus 140 according to the first embodiment can present the treatment plan based on the index value with respect to the coronary artery and the myocardium.

### Second Embodiment

In the above first embodiment, the case in which the ratio between the “Stress FRR” and the “Rest FFR” is calculated as the “INDEX” has been described. In a second embodiment, a case in which the INDEX is calculated without calculating the FFR will be described. Note that the medical image processing apparatus 140 according to the second embodiment differs from that of the first embodiment in the processing contents by the calculating function 145b. Hereinafter, this point will be mainly described.

The calculating function 145b according to the second embodiment calculates the INDEX based on pressure information used for calculating the FFR. As indicated in the above formula (3), the FFR is expressed by the ratio between the pressure “Pa” on the upstream side of the lesion (e.g., the stenosis) and the pressure “Pd” on the downstream side of the lesion (e.g., the stenosis). Thus, the “INDEX” can be expressed by a ratio between a downstream pressure “Pdₛₜ” at stress and a downstream pressure “Pdᵣₑ” at rest as indicated in the following formula (5).

$$INDEX = \frac{Stress FFR}{Rest FFR} = \frac{\frac{{Pd}_{s t} - P {v}_{s t}}{P {a}_{s t} - P {v}_{s t}}}{\frac{P {d}_{r e} - P {v}_{r e}}{P {a}_{r e} - P {v}_{r e}}} \approx \frac{\frac{{Pd}_{s t}}{P {a}_{s t}}}{\frac{{Pd}_{re}}{P {a}_{re}}} \approx \frac{{Pd}_{{s}_{⃛} t}}{{Pd}_{r e}} \qquad ( 5 )$$

That is, as indicated in the formula (5), the “Stress FFR” in the “INDEX” can be replaced with a value obtained by dividing the downstream pressure “Pdₛₜ” at stress by an upstream pressure “Paₛₜ” at stress based on the formula (3). Similarly, the “Rest FFR” in the “INDEX” can be replaced with a value obtained by dividing the downstream pressure “Pdᵣₑ” at rest by an upstream pressure “Paᵣₑ” at rest based on the formula (3).

Moreover, since the upstream pressure “Pa” can be considered to be equal between a stress state and a rest state, the upstream pressure “Paₛₜ” at stress and the upstream pressure “Paᵣₑ” at rest can be deleted. As a result, the “INDEX” can be calculated by the ratio between the downstream pressure “Pdₛₜ” at stress and the downstream pressure “Pdᵣₑ” at rest as indicated in the formula (5).

Thus, the calculating function 145b according to the second embodiment calculates the ratio between the downstream pressure “Pdₛₜ” at stress and the downstream pressure “Pdᵣₑ” at rest as the “INDEX” based on the formula (5).

As described above, according to the second embodiment, the calculating function 145b calculates the INDEX based on the pressure information used for calculating the FFR. Thus, the medical image processing apparatus 140 according to the second embodiment can calculate the INDEX by using the pressure “Pd” on the downstream side of the lesion calculated during the calculation of the FFR, thereby reducing calculation costs.

### Third Embodiment

In the above first embodiment, the case in which the disease is determined by the “INDEX” has been described. In a third embodiment, a case in which the disease is determined by combining the “INDEX” and the “CFR” will be described. Note that the medical image processing apparatus 140 according to the third embodiment differs from that of the first embodiment in the processing contents by the acquiring function 145a and the processing contents by the calculating function 145b. Hereinafter, this point will be mainly described.

The medical image processing apparatus 140 according to the third embodiment displays information regarding a disease based on the “INDEX” and the “CFR”. In such a case, the acquiring function 145a according to the third embodiment acquires the coronary flow reserve of the subject. For example, the acquiring function 145a acquires image data from the X-ray CT apparatus 110 or the medical image storage apparatus 120 via the NW interface 141, and calculates the CFR based on the acquired image data. Note that the CFR is calculated as appropriate by a known method. The acquiring function 145a can also acquire the CFR value of the subject acquired by another medical apparatus via a network.

The display control function 145d according to the third embodiment displays the information regarding a disease based on the “INDEX” and the “CFR”. For example, the display control function 145d determines a disease possibility based on the comparison result between the “INDEX” and the normal range and a comparison result between the “CFR” and a reference value, and displays the determination result.

For example, the display control function 145d compares the CFR value with the reference value (threshold), and determines that the coronary artery or the myocardium is abnormal when the CFR value falls below the reference value. When the CFR value falls below the reference value and the INDEX exceeds the normal range, the display control function 145d determines that the myocardium is abnormal, and displays information indicating that the myocardium has a disease possibility. Additionally, when the CFR value falls below the reference value and the INDEX falls below the normal range, the display control function 145d determines that the coronary artery is abnormal, and displays information indicating that the coronary artery has a disease possibility.

When the CFR value falls below the reference value and the INDEX is within the normal range, the display control function 145d displays information indicating that either the myocardium or the coronary artery has a disease possibility but it is not possible to determine which of them has a disease possibility.

The medical image processing apparatus 140 according to the third embodiment also calculates an index value different from the INDEX based on the “INDEX” and the “CFR”, and displays the information regarding a disease based on the calculated index value. In such a case, the acquiring function 145a acquires the CFR value of the subject similarly to the above case.

The calculating function 145b according to the third embodiment calculates a second index value based on a first index value as the INDEX, and the CFR. For example, the calculating function 145b calculates an index “INDEX2” by multiplying a coefficient “A” and a coefficient “B” together as indicated in the following formula (6).

INDEX2=A×B (6)

Note that the coefficient “A” in the formula (6) indicates an absolute value of a difference between the “CFR” and the reference value. Additionally, the coefficient “B” indicates a degree of the “INDEX” deviating from the normal range. The degree of the “INDEX” deviating from the normal range is defined as a minus value when the INDEX value is below the normal range, and as a plus value when the INDEX value is above the normal range.

The display control function 145d displays the information regarding a disease based on the “INDEX2”. For example, when the “INDEX2” value calculated by the calculating function 145b is a large plus value, the display control function 145d determines that the myocardium is abnormal, and displays information indicating that the myocardium has a disease possibility. Meanwhile, when the “INDEX2” value calculated by the calculating function 145b is a large minus value, the display control function 145d determines that the coronary artery is abnormal, and displays information indicating that the coronary artery has a disease possibility.

As described above, according to the third embodiment, the acquiring function 145a acquires the CFR value of the subject. The display control function 145d displays the information regarding the disease based on the INDEX and the CFR. Thus, the medical image processing apparatus 140 according to the third embodiment can present the information regarding the disease possibility by combining the CFR and the INDEX.

According to the third embodiment, the calculating function 145b calculates the “INDEX2” based on the first index value as the INDEX, and the CFR. Thus, the medical image processing apparatus 140 according to the third embodiment can calculate the index by combining the CFR and the INDEX.

According to the third embodiment, the display control function 145d displays the information regarding the disease based on the INDEX2. Thus, the medical image processing apparatus 140 according to the third embodiment can determine the disease possibility based on the index obtained by combining the CFR and the INDEX, and present the determination result.

### OTHER EMBODIMENTS

While the case in which the coronary artery CT image is used as the medical image regarding the coronary artery has been described in the above embodiments, the embodiments are not limited thereto. For example, any type of medical image that enables calculation of a blood vessel shape and flow information such as a blood flow velocity may be used. For instance, an ultrasonic image obtained by an ultrasonic diagnostic image or an MR image obtained by an MRI apparatus may be used.

The case in which the “Stress FFR” and the “Rest FFR” are acquired by using the coronary artery CT image collected from the subject at rest has been described in the above embodiments. However, the embodiments are not limited thereto. The “Stress FFR” may be acquired by using the coronary artery CT image collected from the subject at stress, and the “Rest FFR” may be acquired by using the coronary artery CT image collected from the subject at rest.

Additionally, the “Stress FFR” and the “Rest FFR” may be acquired by inserting a pressure wire into the coronary artery and measuring a pressure at adenosine stress and a pressure at no stress. In such a case, the acquiring function 145a calculates the “Stress FFR” and the “Rest FFR” based on the pressures acquired by the pressure wire.

While the case in which the information regarding the FFR and the INDEX is displayed on the display 144 of the medical image processing apparatus 140 has been described in the above embodiments, the embodiments are not limited thereto. For example, the information regarding the FFR and the INDEX may be displayed on the display of the medical information display apparatus 130.

While the case in which the acquiring unit, the calculating unit, and the display control unit in the present specification are achieved by the acquiring function, the calculating function, and the display control function of the processing circuitry, respectively, has been described in the above embodiments, the embodiments are not limited thereto. For example, the functions of the acquiring unit, the calculating unit, and the display control unit in the present specification may be achieved by only hardware, only software, or a combination of hardware and software as well as being achieved by the acquiring function, the calculating function, and the display control function as described in the embodiments.

For example, the term “processor” used in the above embodiments indicates a central processing unit (CPU), a graphics processing unit (CPU), or a circuit such as an application specific integrated circuit (ASIC) and a programmable logic device (e.g., a simple programmable logic device (SPLD), a complex programmable logic device (CPLD), and a field programmable gate array (FPGA)). Instead of storing the computer programs in the memory circuit, the computer programs may be directly incorporated in the circuit of the processor. In this case, the processor reads and executes the computer programs incorporated in its circuit to implement the functions. The respective processors described in the above embodiments are not limited to single-circuit processors. A plurality of independent circuits may be combined and integrated as one processor to implement the functions.

The computer programs executed by the processor are provided by being incorporated in a read-only memory (ROM), a memory circuit, or the like in advance. Note that the computer programs may be provided by being recorded on a non-transitory computer-readable recording medium such as a compact disc-read only memory (CD-ROM), a flexible disk (FD), a compact disc-recordable (CD-R), and a digital versatile disc (DVD), as files in an installable or executable format in the device. The computer programs can be also stored in a computer connected to a network such as the Internet, and provided or distributed by being downloaded via the network. For example, the computer programs have a module configuration including each processing function described above. As actual hardware, the CPU reads and executes the computer programs from the recording medium such as the ROM, thereby loading each module onto the main memory, and generating each module on the main memory.

Moreover, in the embodiments and modifications described above, each component of each apparatus illustrated in the drawings is a functional concept, and these components are not necessarily constituted physically as illustrated in the drawings. In other words, the specific configuration of dispersion/integration of each apparatus is not limited to the illustrated configuration, and all or a part of each apparatus may be dispersed or integrated functionally or physically in an optional unit in accordance with various types of loads or operating conditions. Furthermore, all or a part of each processing function performed by each apparatus can be achieved by a CPU and a computer program analyzed and executed by the CPU, or can be achieved as hardware by wired logic.

Moreover, among the processes described in the embodiments and modifications described above, all or a part of processes described as being automatically performed can be manually performed. Alternatively, all or a part of processes described as being manually performed can be automatically performed using a known method. Also, processing procedures, control procedures, specific titles, and information including various types of data and parameters, which are described above and illustrated in the drawings, may be optionally changed unless otherwise specified.

According to at least one of the embodiments described above, the index for the myocardial function can be provided.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured to:
acquire fractional flow reserve at rest in a coronary artery of a subject, and fractional flow reserve at stress in the coronary artery;
calculate an index value based on comparison between the fractional flow reserve at rest and the fractional flow reserve at stress; and
display the index value as an index for a function of myocardium of the subject.

2. The medical image processing apparatus according to claim 1, wherein
the processing circuitry is configured to acquire the fractional flow reserve at rest and the fractional flow reserve at stress by computational fluid dynamics using a medical image including the coronary artery of the subject.

3. The medical image processing apparatus according to claim 1, wherein
the processing circuitry is configured to calculate a ratio between the fractional flow reserve at rest and the fractional flow reserve at stress.

4. The medical image processing apparatus according to claim 1, wherein
the processing circuitry is configured to compare the index value calculated at each position of the coronary artery of the subject with a threshold, identify a position on the coronary artery where the index value exceeds the threshold, and display a dominant region on the myocardium corresponding to the identified position.

5. The medical image processing apparatus according to claim 4, wherein
the processing circuitry is configured to identify a position closest to a proximal where the index value exceeds the threshold in the positions of the coronary artery of the subject, and display a dominant region on the myocardium corresponding to the identified position.

6. The medical image processing apparatus according to claim 5, wherein
the processing circuitry is configured to display a warning when an area of the dominant region exceeds a threshold.

7. The medical image processing apparatus according to claim 1, wherein
the processing circuitry is configured to compare the index value calculated at each position of the coronary artery of the subject with a numerical value range, identify a position on the coronary artery where the index value exceeds the numerical value range, or a position on the coronary artery where the index value falls below the numerical value range, and display display information based on a comparison result.

8. The medical image processing apparatus according to claim 7, wherein
the processing circuitry is configured to display disease candidates individually corresponding to a case in which the index value exceeds the numerical value range and a case in which the index value falls below the numerical value range.

9. The medical image processing apparatus according to claim 8, wherein
the processing circuitry is configured to display a disease candidate for the myocardium when the index value exceeds the numerical value range, and display a disease candidate for the coronary artery when the index value falls below the numerical value range.

10. The medical image processing apparatus according to claim 1, wherein
the processing circuitry is configured to display a combination of treatment for the coronary artery of the subject and treatment for the myocardium of the subject based on a comparison result between the fractional flow reserve at rest or the fractional flow reserve at stress and a first threshold, and a comparison result between the index value and a second threshold.

11. The medical image processing apparatus according to claim 1, wherein
the processing circuitry is configured to acquire coronary flow reserve of the subject, and display information regarding a disease based on the index value and the coronary flow reserve.

12. The medical image processing apparatus according to claim 11, wherein
the processing circuitry is configured to calculate a second index value based on a first index value as the index value, and the coronary flow reserve.

13. The medical image processing apparatus according to claim 12, wherein
the processing circuitry is configured to display the information regarding a disease based on the second index value.

14. A medical image processing system comprising: a medical image processing apparatus and a medical information display apparatus,
the medical image processing apparatus comprising processing circuitry configured to:
acquire fractional flow reserve at rest in a coronary artery of a subject, and fractional flow reserve at stress in the coronary artery;
calculate an index value based on comparison between the fractional flow reserve at rest and the fractional flow reserve at stress; and
display the index value as an index for a function of myocardium of the subject.

15. A medical image processing method comprising:
acquiring fractional flow reserve at rest in a coronary artery of a subject, and fractional flow reserve at stress in the coronary artery;
calculating an index value based on comparison between the fractional flow reserve at rest and the fractional flow reserve at stress; and
displaying the index value as an index for a function of myocardium of the subject.
